(19) United States
(12) Patent Application Publication
(10) Pub. No.: US 2004/0241198 A1
(43) Pub. Date: Dec. 2, 2004

(54) Cosmetic composition comprising at least one polyester resulting from esterification of at least one triglyceride of hydroxylated carboxylic acid(s) and at least one oil with a molar mass of 650 to 10 000 g/mol

(76) Inventors: Xavier Blin, Paris (FR); Vanina Filippi, Paris (FR)

Correspondence Address: Thomas L. Irving, FINNEGAN, HENDERSON, FARABOW, GARRETT, & DUNNER, L.L.P., 1300 I Street, N.W., Washington, DC, 20005-3315, US

(21) Appl. No.: 10/796,979

(22) Filed: Mar. 11, 2004

Related U.S. Application Data
(60) Provisional application No. 60/458,371, filed on Mar. 31, 2003.

(30) Foreign Application Priority Data
Mar. 12, 2003 (FR) 03 03078

(51) Int. Cl. C11D 3/00; A61K 7/00

(52) U.S. Cl. 424/401

(57) ABSTRACT

Disclosed herein is a composition, for example, a cosmetic care and/or makeup composition for a keratin material, comprising, in a cosmetically acceptable medium, i) at least one polyester resulting from esterification of at least one triglyceride of at least one hydroxylated carboxylic acid with at least one aliphatic monocarboxylic acid and at least one aliphatic dicarboxylic acid, ii) at least one oil of a molar mass ranging from 650 to 10 000 g/mol, and iii) at least one colorant. The composition may possess cosmetic properties and can, for example, endow the makeup or care product with properties of gloss, smoothness of application and comfort. Further disclosed herein is the use of the at least one polyester and the at least one oil with a molar mass ranging from 650 to 10 000 g/mol in a physiologically acceptable composition as an agent for endowing the composition with properties of slip, gloss, comfort, definition, non-migration, enhanced color intensity and/or enhanced color retention after challenge.

This application claims benefit of U.S. Provisional Application No. 60/458,371, filed Mar. 31, 2003.

Disclosed herein is a cosmetic makeup and/or care composition for skin, including scalp, of both face and body, lips or the epidermal derivatives of humans, such as hair, eyelashes, eyebrows and nails, which comprises, in a cosmetically acceptable medium, at least one specific polyester. This composition can have notable cosmetic properties and can endow the makeup or care product with at least one property chosen from properties, for example, of slip, gloss, comfort, outline definition, non-migration, enhanced color intensity and enhanced color retention after challenge.

In one embodiment, the composition disclosed herein may constitute a makeup product for the body, lips or epidermal derivatives of humans which may have properties, for example, of non-therapeutic treatment and/or care. For example, the composition disclosed herein may constitute a lipstick or lipgloss, a blusher or eyeshadow, a tattooing product, a mascara, an eyeliner, a nail varnish, an artificial tanning product for the skin or a haircare or hair coloring product.

Numerous cosmetic compositions exist for which gloss properties in the deposited film are desirable following its application to keratin materials (such as skin, lips, epidermal derivatives). Examples that may be mentioned include lipsticks, nail varnishes and certain hair products.

From this viewpoint, a formulator may have a number of types of base materials at his or her disposal, such as lanolins, which may be used in combination with what are termed as “gloss” oils, such as a) polybutenes, which have a high viscosity, b) fatty alcohol or acid esters with a high carbon number (typically greater than 16), c) certain vegetable oils, d) esters resulting from partial or complete esterification of a hydroxylated aliphatic compound with an aromatic acid, as described in patent application EP 1 097 699, and e) polyesters obtained by sequential reaction of castor oil with isostearic acid and then with succinic acid, which are described in U.S. Pat. No. 6,342,527.

In one embodiment, disclosed herein is a glossy cosmetic care and/or makeup composition for keratin materials, such as skin, lips and epidermal derivatives, which can have improved properties relative to the cosmetic compositions of prior art. For example, deposition of the composition disclosed herein on the keratin materials can be more sharply defined and its color retention can be enhanced.

In one embodiment, the present inventors have found that the use i) of at least one polyester resulting from esterification of at least one triglyceride of at least one hydroxylated aliphatic acid with at least one aliphatic monocarboxylic acid and at least one aliphatic dicarboxylic acid, in combination with ii) at least one (i.e., one or more than one) oil with a molar mass ranging from 650 to 10 000 g/mol, makes it possible to obtain a cosmetic composition which can be glossy on application and over time, can exhibit at least one property chosen from good application properties, good properties of spreading, of color retention after challenge, of comfort (no tightening or drying out) and of non-migration and whose outlines, when deposited on keratin materials, can be well defined and whose color intensity can be enhanced.

The oil with a molar mass ranging from 650 to 10 000 g/mol is referred to herein as an oil of high molar mass.

In one embodiment, disclosed herein is a composition comprising, in a cosmetically acceptable medium, i) at least one polyester resulting from esterification of at least one triglyceride of at least one hydroxylated carboxylic acid with at least one aliphatic monocarboxylic acid and at least one aliphatic dicarboxylic acid, ii) at least one oil of a molar mass ranging from 650 to 10 000 g/mol, and iii) at least one colorant.

In another embodiment, disclosed herein is a cosmetic method of endowing a film of a cosmetic composition with at least one property chosen from gloss on application and over time, good application properties and good properties of spreading, of color retention following challenge, of comfort (no tightening or drying out) and of non-migration, with outlines which, when deposited on keratin materials, are well defined, and with enhanced color intensity, wherein the method comprises including in the composition i) at least one polyester resulting from esterification of at least one triglyceride of at least one hydroxylated carboxylic acid with at least one aliphatic monocarboxylic acid and at least one aliphatic dicarboxylic acid, ii) at least one oil of a molar mass ranging from 650 to 10 000 g/mol, and iii) at least one colorant.

In yet another embodiment, disclosed herein is the use of the combination of i) at least one polyester resulting from esterification of at least one triglyceride of at least one hydroxylated carboxylic acid with at least one aliphatic monocarboxylic acid and at least one aliphatic dicarboxylic acid and ii) at least one oil of a molar mass ranging from 650 to 10 000 g/mol, in a physiologically acceptable composition comprising at least one colorant for endowing the composition with at least one property chosen from gloss on application and over time, good application properties and good properties of spreading, of outline definition, of comfort (no tightening or drying out) and of non-migration, with enhanced color intensity and with enhanced color retention after challenge.

In one embodiment, the composition disclosed herein is free of lanolin or lanolin derivatives.

As disclosed herein, the term “lanolin derivatives” means, for example, liquid lanolin, reduced lanolin, lanolin purified by adsorption, lanolin acetate, lanolin wax, for example the oxypropylenated (5 PO) lanolin wax sold as Emery 1695 by Cognis, isopropyl lanolate, liquid lanolin acetate, hydroxylanolin, polyoxyethylene-lanolin, lanolin fatty acid, hard lanolin fatty acid, cholesteryl esters of lanolin fatty acid, lanolin alcohol, and lanolin alcohol acetate.

In another embodiment, the composition disclosed herein comprises at least one pasty compound other than lanolin derivatives.

Polyester from at Least One Triglyceride of at Least One Hydroxylated Aliphatic Acid

The composition disclosed herein comprises at least one polyester resulting from esterification of at least one triglyceride of at least one hydroxylated carboxylic acid with at least one aliphatic monocarboxylic acid and at least one aliphatic dicarboxylic acid, which is optionally unsaturated.

In one embodiment, the at least one hydroxylated carboxylic acid disclosed herein is chosen from aliphatic hydroxycarboxylic acids. The at least one triglyceride of at least one hydroxylated carboxylic acid disclosed herein may be, for example, a glycerol substituted by three hydroxylated carboxylic acid residues, which can be identical or different. For example, a triglyceride obtained by reacting one equivalent of glycerol and three equivalents of a hydroxylated carboxylic acid can be called “triglyceride of hydroxylated carboxylic acid”. A triglyceride obtained by reacting one equivalent of glycerol with three equivalents of a mixture of at least two different hydroxylated carboxylic acids can be called “triglyceride of hydroxylated carboxylic acid(s)”. Both types belong to the category “triglyceride of at least one hydroxylated carboxylic acid” as disclosed herein.

In one embodiment, the at least one polyester disclosed herein is liquid at ambient temperature (generally ranging from 20° C. to 25° C.) and atmospheric pressure (760 mm Hg).

In another embodiment, the at least one polyester disclosed herein has a viscosity at 25° C. of more than 500 cP (50 Pa.s), such as ranging from 900 to 10 000 cP (90 to 1 000 Pa.s) and further such as from 950 to 5 000 cP (95 to 500 Pa.s). Viscosity, as used herein, is measured with a Brookfield RV or Brookfield “DV-II+” viscometer of type LV equipped with a No.1 spindle rotating at a speed ranging from 0.5 rpm to 10 rpm. This viscosity measurement is taken when the measurement value has stabilized, generally after 10 minutes.

In another embodiment, the at least one polyester disclosed herein has a refractive index greater than or equal to 1.47, such as from 1.47 to 1.55, and further such as from 1.48 to 1.55 (wherein the refractive index is defined for the sodium D line). The refractive index is measured at 20° C. by reference to D Ray sodium (589 nm) according to, for example, ASTM Standards D 1218-92, D1500.

According to one embodiment, the at least one polyester is obtained by two esterification reactions of at least one triglyceride of at least one hydroxylated carboxylic acid: one esterification with at least one aliphatic monocarboxylic acid and one esterification with at least one aliphatic dicarboxylic acid.

In this embodiment, the at least one polyester is obtained by

a) esterifying at least one hydroxyl functional group of at least one triglyceride of at least one hydroxylated carboxylic acid with at least one aliphatic monocarboxylic acid and

b) esterifying the remaining hydroxyl functional groups of the at least one triglyceride of at least one hydroxylated carboxylic acid, i.e., those not esterified with the at least one aliphatic monocarboxylic acid, with at least one aliphatic dicarboxylic acid.

The esterification with at least one aliphatic monocarboxylic acid may be, for example, conducted before the esterification with at least one aliphatic dicarboxylic acid.

The at least one hydroxylated carboxylic acid (i.e., the precursor of the triglyceride of at least one hydroxylated carboxylic acid) is, for example, chosen from hydroxylated aliphatic carboxylic acids comprising from 6 to 40 carbon atoms, such as from 10 to 34 carbon atoms and further such as from 12 to 28 carbon atoms, and even further such as from 16 to 20 carbon atoms. In one embodiment, the at least one hydroxylated carboxylic acid comprises 18 carbon atoms.

The at least one hydroxylated carboxylic acid is, for example, chosen from saturated and unsaturated fatty acids.

For example, the at least one hydroxylated carboxylic acid may be chosen from

i) saturated linear monohydroxylated aliphatic monocarboxylic acids of formulae (1) and (2)

(1)

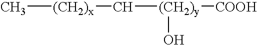

with 3≦x+y≦37

and

HO—CH₂—(CH₂)ₓ—COOH (2)

with 4≦x≦38;

ii) saturated branched monohydroxylated aliphatic monocarboxylic acids of formulae (3) and (3′)

(3)

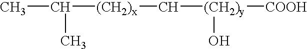

with 1≦x+y≦35

and (3′) 2-ethyl-3-hydroxycaprylic acid of formula

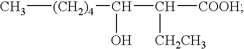

iii) unsaturated monohydroxylated aliphatic monocarboxylic acids of formulae (4), (5), and (6)

(4)

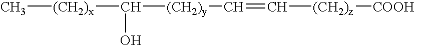

with 1≦x+y+z≦35

(5)

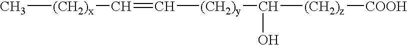

with 1≦x+y+z≦35

and

HOCH₂—(CH₂)ₓ—CH═CH—(CH₂)_{y}—COOH (6)

with 2≦x+y≦36;

iv) saturated polyhydroxylated aliphatic monocarboxylic acids of formula (7)

(7)

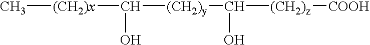

with 2≦x+y+z≦36

and the corresponding unsaturated polyhydroxylated aliphatic monocarboxylic acids;

v) saturated monohydroxylated aliphatic polyacids of formula (8)

(8)

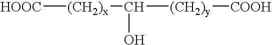

with 3≦x+y≦37

and the corresponding unsaturated monohydroxylated aliphatic polyacids; and

vi) saturated and unsaturated polyhydroxylated aliphatic polyacids.

In one embodiment, the at least one hydroxylated carboxylic acid is chosen from

12-hydroxystearic acid, α-hydroxyoctadecanoic acid, hydroxy-14-eicosenoic acid;

leucinic acid, 2-ethyl-3-hydroxycaprylic acid;

ricinoleic acid;

hydroxy-4-hexanoic acid, oxynervonic acid;

16-hydroxy-6-hexadecenoic acid; and

9,10-dihydroxyoctadecanoic acid, 9,12-dihydroxyoctadecanoic acid, aleuritic acid,

9,10,12-trihydroxyoctadecanoic acid, hexahydroxyoctadecanoic acid and octahydroxyoctadecanoic acid.

The at least one hydroxylated carboxylic acid may, for example, be chosen from unsaturated fatty acids comprising from 16 to 20 carbon atoms, such as 18 carbon atoms.

The at least one triglyceride disclosed herein is, for example, triglyceride of ricinoleic acid. This triglyceride is present in large amounts in the natural state in castor oil.

The at least one triglyceride of at least one hydroxylated carboxylic acid disclosed herein may, for example, be chosen from triglycerides of at least one hydroxylated carboxylic acid such as the at least one hydroxylated carboxylic acid comprises from 6 to 40 carbon atoms, such as from 10 to 34 carbon atoms, and further such as from 12 to 28 carbon atoms, and even further such as from 16 to 20 carbon atoms. In one embodiment, the at least one hydroxylated carboxylic acid comprises 18 carbon atoms.

The at least one aliphatic monocarboxylic acid disclosed herein may be chosen from saturated and unsaturated aliphatic fatty acids, such as isostearic acid.

The at least one aliphatic dicarboxylic acid disclosed herein may comprise, for example, from 3 to 10 carbon atoms, such as from 3 to 6 carbon atoms, and further such as from 3 to 5 carbon atoms. According to one embodiment, the at least one aliphatic dicarboxylic acid is chosen from those of formula HOOC—(CH₂)ₙ—COOH wherein n=1 to 4.

For example, the at least one aliphatic dicarboxylic acid is succinic acid, corresponding to the above formula wherein n=2.

In one embodiment, the at least one polyester disclosed herein is of the formula (I)

T₂O—(OC-D-CO—O-T₁-O)ₓ—OC-D-CO-OT₂ (I)

wherein

The T₂-O— and —O-T₂ portions of the at least one polyester are derived from T₂-OH, which is a triglyceride of at least one hydroxylated carboxylic acid, wherein the triglyceride has been esterified with two molecules of at least one aliphatic monocarboxylic acid, and the triglyceride comprises a single free hydroxyl functional group;

The O-T₁-O— portion of the at least one polyester is derived from OH-T₁-OH, which is a triglyceride of at least one hydroxylated carboxylic acid, wherein the triglyceride has been esterified with one molecule of at least one aliphatic monocarboxylic acid, and the triglyceride comprises two free hydroxyl functional groups;

The —OC-D-CO— portion of the at least one polyester is derived from HOOC-D-COOH, which is the at least one dicarboxylic acid; and

x ranges from 1 to 50, such as from 1 to 10, and further such as from 2 to 6.

For example, x can be chosen from 3, 4, 5, 6, 7, 8, 9, and 10.

The at least one polyester disclosed herein can be chosen, for example, from the polyesters described in U.S. Pat. No. 6,342,527, the content of which is incorporated by reference into the present application. The at least one polyester disclosed herein can, for example, be of the formula (I) above wherein

T₂O— is

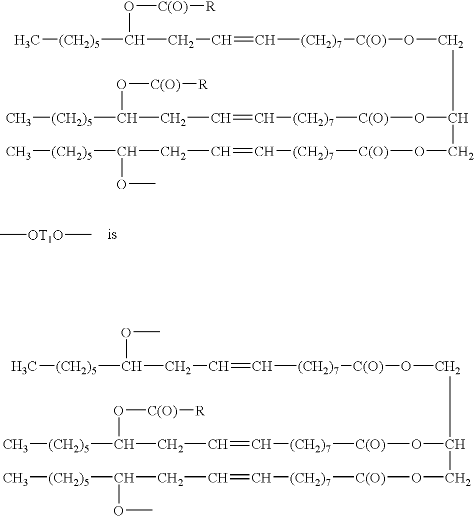

In the above two formulae, R is chosen from alkyl and alkylene groups comprising from 5 to 33 carbon atoms.

In one embodiment, R is chosen from alkyl groups comprising from 7 to 17 carbon atoms alkylene groups comprising from 11 to 21 carbon atoms.

The at least one polyester disclosed herein may be present in an amount ranging from 0.1% to 99.9% by weight, such as from 1% to 99% by weight, and further such as from 1% to 80% by weight, such as from 10% to 40% by weight, and further such as from 15% to 25% by weight, even further such as from 20% to 25% by weight of the total weight of the composition. In general, the at least one polyester can be present in an amount sufficient to endow the composition with at least one property chosen from properties of gloss, stability, color retention over time, gloss retention, comfort, non-migration and outline definition following an application of the composition.

High Molecular Mass Oil

In one embodiment, the composition disclosed herein further comprises at least one oil of high molar mass, ranging from 650 to 10.000 g/mol. As disclosed herein, an oil is a non-aqueous, water-immiscible compound, which is liquid at ambient temperature (25° C.) and atmospheric pressure (760 mm Hg).

The at least one oil used in the composition disclosed herein may have a molar mass ranging from 650 to 10 000 g/mol, such as from 750 to 7 500 g/mol.

Oils with a molar mass, which is too low, when combined with the at least one polyester disclosed herein, may lead to compositions, which lack sufficient gloss. On the other hand, oils with a molar mass, which is too high, may give rise to compositions, which are considered to be excessively sticky.

For example in some applications, the triglyceride of capric/caprylic acids (such as that sold or manufactured as Estol 3603 MCT oil by Uniqema), which has a molar mass of 494 g/mol, may lead to compositions whose cosmetic properties are inferior to those of the composition disclosed herein.

The at least one oil with a molar mass ranging from 650 to 10 000 g/mol that can be used herein may be chosen, for example, from

lipophilic polymers such as

polybutylenes such as Indopol H-100 (with a molar mass MM=965 g/mol), Indopol H-300 (MM=1340 g/mol) and Indopol H-1 500 (MM=2160 g/mol), which are sold or manufactured by Amoco,

hydrogenated polyisobutylenes such as Panalane H-300 E, sold or manufactured by Amoco (M=1 340 g/mol), Viseal 20000, sold or manufactured by Synteal (MM=6 000 g/mol) and Rewopal PIB 1000, sold or manufactured by Witco (MM=1 000 g/mol),

polydecenes and hydrogenated polydecenes such as Puresyn 10 (MM=723 g/mol) and Puresyn 150 (MM=9 200 g/mol), sold or manufactured by Mobil Chemicals, and

vinylpyrrolidone copolymers such as the vinylpyrrolidone/1-hexadecene copolymer Antaron V-216, sold or manufactured by ISP (MM=7 300 g/mol);

esters such as:

linear fatty acid esters comprising a total carbon number ranging from 35 to 70, such as pentaerythrityl tetrapelargonate (MM=697 g/mol),

hydroxylated esters such as polyglycerol-2 triisostearate (MM=965 g/mol),

aromatic esters such as tridecyl trimellitate (MM=757 g/mol), and

branched C₂₄-C₂₈ fatty acid and fatty alcohol esters such as those described in document EP-A-0 955 039, such as triisoarachidyl citrate (MM 1 033.76 g/mol), pentaerythrityl tetraisononanoate. (MM=697 g/mol), glyceryl triisostearate (MM=891 g/mol), glyceryl 2-tridecyltetradecanoate (MM=1143 g/mol), pentaerythrityl tetraisostearate (MM=1 202 g/mol), polyglyceryl-2 tetraisostearate (MM=1 232 g/mol) and pentaerythrityl 2-tetradecyltetradecanoate (MM=1 538 g/mol);

silicone oils such as phenylsilicones, for instance Belsil PDM 1000 from Wacker (MM=9 000 g/mol);

oils of vegetable origin such as sesame oil (820 g/mol);

and mixtures thereof.

In one embodiment, the at least one oil with a molar mass ranging from 650 to 10 000 g/mol that is used in the composition disclosed herein may be present in an amount ranging from 1% to 99% by weight, such as from 10 to 80% by weight, and further such as from 5% to 70% by weight of the total weight of the composition.

The composition disclosed herein may further comprise at least one colorant, which may be chosen from dyes which are soluble or dispersible in the composition, pigments, and nacres. The dyes may, for example, be chosen from fat-soluble dyes, although water-soluble dyes can also be used. The at least one colorant may be present in an amount ranging from 0.001% to 98% by weight, such as from 0.5% to 85% by weight, and further such as from 1% to 60% by weight of the total weight of the composition.

For a composition in paste or cast form, such as a lipstick or a body makeup product, the at least one colorant is present in an amount ranging, for example, from 0.5% to 50% by weight, such as from 2% to 40% by weight, and further such as from 5% to 30% by weight, relative to the total weight of the composition.

The fat-soluble dyes may, for example, be chosen from Sudan Red, b & C Red 17, D & C Green 6, β-carotene, soya oil, Sudan Brown, D & C Yellow 11, D & C Violet 2, D & C Orange 5, quinoline yellow and annatto. They may be present in an amount ranging from 0 to 20% by weight, such as from 0.1% to 6% by weight of the total weight of the composition. The water-soluble dyes may, for example, be chosen from beet juice and methylene blue, and may be present in an amount ranging from 0.1% to 6% by weight of the total weight of the composition (if they are present).

The composition disclosed herein may, for example, comprise a particulate phase, which may, for example, be colored and may be present in an amount ranging from 0.001% to 50% by weight, such as from 0.01% to 40% by weight, and further such as from 0.05% to 30% by weight of the total weight of the composition. The particulate phase may comprise at least one ingredient chosen from pigments, nacres, and fillers, which are commonly used in cosmetic compositions.

As used herein, the term “pigments” means white or colored, organic or inorganic particles, which are insoluble in a liquid fatty phase and are intended for coloring and/or opacifying the composition. The term “fillers” means colorless or white, mineral or synthetic particles, which may be lamellar or non-lamellar. The tem “nacres” means irridescent particles, which are produced, for example, by certain molluscs in their shells, or are synthesized. These fillers and nacres can serve, for example, to modify the texture of the composition.

The pigments may be present in an amount ranging from 0.05% to 30% by weight (if they are present), such as from 2% to 20% by weight of the total weight of the composition. As mineral pigments, which can be used herein, mention may be made, for example, of titanium oxide, zirconium oxide and cerium oxide and also of zinc oxide, iron oxide, chromium oxide and ferric blue. Among the organic pigments, which can be used herein, mention may be made, for example, of carbon black and lakes of barium, strontium, calcium (D & C Red No. 7) and aluminium.

The nacres may be present in an amount ranging from 0.001% to 20% by weight (if they are present), such as from 1% to 15% by weight of the total weight of the composition. Among the nacres, which can be used herein, mention may be made, for example, of mica covered with titanium dioxide, iron oxide, natural pigment or bismuth oxychloride, such as coloured titanium mica, goniochromatic pigments and, for example, multi-layer interference pigments.

The fillers may be present in an amount ranging from 0.001% to 35% by weight (if they are present), such as from 0.5% to 15% by weight of the total weight of the composition. Mention may be made, for example, of talc, mica, kaolin, Nylon® powder (Orgasol in particular) and polyethylene powder, polytetrafluoroethylene (Teflon®) powders, starch, boron nitride, copolymer microspheres such as Expancel® (Nobel Industries), Polytrap® (Dow Corning), Polypore® L 200 (Chemdal Corporation) and silicone resin microbeads (such as Tospearl® from Toshiba), and silica.

The composition disclosed herein may further comprise at least one additional non-aqueous compound other than the at least one polyester and the at least one oil with a molar mass ranging from 650 to 10 000 g/mol. The at least one additional non-aqueous compound may be chosen, for example, from other oils, pastelike fats, waxes, gums, and resins.

In one embodiment, the composition disclosed herein further comprises at least one wax. As disclosed herein, a wax is a lipophilic fatty compound, which is solid at ambient temperature (25° C.), exhibits a reversible solid/liquid state change, has a melting temperature greater than 30° C. and possibly up to 200° C., has a hardness greater than 0.5 MPa, and exhibits anisotropic crystalline organization in the solid state. By taking the wax to its melting temperature, it is possible to make it miscible with the oils and to form a microscopically homogeneous mixture, but by taking the temperature of the mixture to ambient temperature, the wax is recrystallized in the oils of the mixture.

The at least one wax, which can be used herein, can be chosen from compounds which are solid at room temperature and are intended to provide the composition with structure, such as in the form of a stick. The at least one wax may be chosen from hydrocarbon waxes, fluoro waxes and silicone waxes and may be chosen from those of vegetable, mineral, animal and synthetic in origin. For example, the at least one wax disclosed herein may have a melting temperature of greater than 40° C., such as greater than 45° C.

As the wax which can be used herein, mention may be made of those waxes which are generally used in the cosmetics field: they are, for example, of natural origin, such as beeswax, carnauba wax, candelilia wax, ouricuri wax, Japan wax, cork fibre wax, sugarcane wax, rice wax, montan wax, paraffin, lignite wax, microcrystalline wax, ceresin, ozokerite, and hydrogenated oils such as jojoba oil; synthetic waxes, such as polyethylene waxes obtained from polymerization or copolymerization of ethylene with a weight-average molecular mass ranging from 400 to 800 g/mol and mixtures thereof, Fischer-Tropsch waxes, and esters of fatty acids, such as octacosanyl stearate, glycerides which are solid at 40° C. or at 45° C., silicone waxes such as alkyl- or alkoxydimethicones comprising a chain chosen from alkyl and alkoxy chains comprising from 10 to 45 carbon atoms, poly(di)methylsiloxane esters which are solid at 40° C. and whose ester chain comprises at least 10 carbon atoms; and mixtures thereof.

The gums which can be used herein are generally in solubilized form in an oil; the polymers are solid at ambient temperature and the resins may be liquid or solid at ambient temperature.

The nature and amount of the gums, pastelike substances or waxes are a function of the desired mechanical properties and textures. For example, the at least one wax may be present in an amount ranging from 0.01% to 50% by weight, such as from 2% to 40% by weight, and further such as from 5% to 30% by weight of the total weight of the composition.

The at least one additional oil other than the oils with a molar mass ranging from 650 to 10 000 g/mol may be chosen, for example, from hydrocarbon oils, silicone oils and fluoro oils. These oils may be animal, vegetable, mineral or synthetic in origin. As disclosed herein, a hydrocarbon oil is an oil which comprises primarily atoms of carbon and hydrogen and optionally at least one functional group chosen from hydroxyl, ester, ether and carboxyl functional groups. In one embodiment, the at least one additional oil is chosen from oils of vegetable and synthetic origins.

The at least one additional oil may be present in an amount ranging from 0.01% to 90% by weight, such as from 0.1% to 60% by weight, and further such as from 10% to 55% by weight of the total weight of the composition.

The composition disclosed herein may further comprise at least one complementary additive chosen from those commonly used in the field, such as water, antioxidants, preservatives, neutralizers, lipophilic gelling agents, liquid non-aqueous compounds, aqueous-phase gelling agents, dispersants and cosmetic active materials. These additives, with the exception of water, may be present in an amount ranging from 0.0005% to 20% by weight, such as from 0.001% to 10% by weight of the total weight of the composition. Water may be present in an amount ranging from 0 to 70% by weight, such as from 1% to 50% by weight, and further such as from 1% to 10% by weight of the total weight of the composition.

As the cosmetic active materials which can be used herein, mention may be made, for example, of vitamins A, E, C, B₃ and F, provitamins such as D-panthenol, active soothing agents such as α-bisabolol, aloe vera, allantoin, plant extracts and essential oils, protective and restructuring agents such as ceramides, active freshness agents such as menthol and its derivatives, emollients (cocoa butter, dimethicone), moisturizers (arginine PCA), active anti-wrinkle substances, essential fatty acids, sunscreens, and mixtures thereof.

The person skilled in the art can take care to select the nature and/or quantity of any complementary additives such that the advantageous properties of the composition disclosed herein are not, or not substantially, adversely affected by the addition envisaged.

The applications of the compositions disclosed herein can be manyfold and pertain to the entirety of cosmetic products, colored or non-colored, such as lipsticks.

The composition disclosed herein may be in the form of a solid composition, compacted or cast, such as in the form of a stick or dish, or in paste or liquid form. In one embodiment, the composition disclosed herein is present in a solid form, such as in a hard form (a form which does not flow under its own weight), for example, a cast or compact form, such as a stick or dish.

The composition disclosed herein may be present in the form of a paste, solid or cream. It may be an oil-in-water or water-in-oil emulsion, an anhydrous gel which is solid or flexible, or in the form of a loose or compacted powder, and even in a two-phase form. In one embodiment, the composition disclosed herein is in the form of a composition comprising an oily, such as anhydrous, continuous phase; in this case, the composition may comprise an amount of water of less than 5% by weight, such as less than 1% by weight of the total weight of the composition.

The composition disclosed herein may be in the form of a colored or non-colored skincare composition, in the form of a sun protection composition or makeup remover composition, or in the form of a hygiene composition. If the composition disclosed herein comprises at least one cosmetic active material, it may then be used as a non-therapeutic treatment or care base for the skin, such as the hands or face, or for the lips (lip balms, protecting the lips from cold, sun and/or wind), or an artificial tanning product for the skin.

The composition disclosed herein may also be in the form of a colored skin makeup product, for example, a face makeup product such as a blusher, rouge or eyeshadow, a body makeup product such as a semi-permanent tattooing product, or a lip makeup product such as a lipstick or lipgloss, possibly having non-therapeutic treatment or care properties, a product for making up the epidermal derivatives, such as a nail varnish, mascara or eyeliner, or a haircare or hair colouring product.

In one embodiment, the composition disclosed herein is in the form of a lipstick or lipgloss.

In one embodiment, the composition disclosed herein should be physiologically acceptable (for example, cosmetically acceptable), i.e., it should be non-toxic and capable of application to the skin, epidermal derivatives or lips of human beings.

The term “cosmetically acceptable medium” means the medium comprises at least one property chosen from being pleasant in taste, feel, appearance and odor and capable of being applied for a number of days in the course of a number of months.

The composition disclosed herein may be manufactured by known methods which are generally employed in the cosmetic field.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The amounts are given as percentages by weight of the total weight of the composition.

### EXAMPLE 1

### Lipstick

| | |
|---|---|
| Castor oil ester of succinic acid and isostearic acid (sold as | 22 |
| Zenigloss by Zenitech) | |
| Ethers of dodecanediol (22 mol) and of polyethylene glycol | 11 |
| (45 EO) (sold as Elfacos ST9 by Akzo Nobel) | |
| Triglyceride of 2-decyltetradecanoic acid | 20 |
| Hydrogenated polyisobutene | 10 |
| Diisostearyl malate | 11 |
| Polybutylene | 2.5 |
| Octacosanyl stearate | 5 |
| Mixture of triglycerides of lauric, myristic, palmitic and | 2 |
| stearic acids (50/20/10/10) | |
| Polyethylene wax | 5 |
| Hectorite □omogeni with distearyl dimethylammonium | 3 |
| chloride | |
| Pigments | qs |
| Preservative | qs |
| Fragrance | qs |

The oily phase was produced by mixing the preservative, all the oils and the paste (ethers of dodecanediol (22 mol) and of polyethylene glycol (45 EO)).

Then the hectorite was ground in the oily phase on a triple-roll mill.

The pigments were subsequently ground in the mixture of the hectorite and the oily phase.

The resulting mixture was added to a pan together with the waxes and the mixture was heated at 105° C. for two hours, with homogenization using a Rayneri apparatus.

Finally the fragrance was added, the mixture was homogenized for 5 minutes and then it was poured into a mold at 42° C. which was cooled at −20° C. for 30 minutes. The sticks were then demolded.

The above formula exhibited good gloss retention at 1 hour and did not migrate at 1 hour. It additionally possessed good properties in terms of application (slip), comfort, gloss (on application and over time) and color retention following challenge.

### EXAMPLE 2

### Comparative

The above formula was produced again, but replacing the castor oil ester of succinic acid and isostearic acid by the castor oil ester of benzoic acid (sold as Finsolv BCO 115 by Finetex).

The composition according to the present disclosure showed better line definition and its color retention following challenge was greater than that of the comparative composition comprising a glossy oil such as the ester of castor oil and benzoic acid. Moreover, the composition according to the present disclosure went on more thickly.

What is claimed is:

1. A composition, comprising, in a cosmetically acceptable medium,
i) at least one polyester resulting from esterification of at least one triglyceride of at least one hydroxylated carboxylic acid with
a) at least one aliphatic monocarboxylic acid and
b) at least one aliphatic dicarboxylic acid,
ii) at least one oil of a molar mass ranging from 650 to 10 000 g/mol, and
iii) at least one colorant.

2. The composition according to claim 1, wherein said at least one polyester is obtained by
a) esterifying at least one hydroxyl functional group of said at least one triglyceride of at least one hydroxylated carboxylic acid with said at least one aliphatic monocarboxylic acid and
b) esterifying the remaining hydroxyl functional groups of said at least one triglyceride of at least one hydroxylated carboxylic acid with said at least one alphatic dicarboxylic acid.

3. The composition according to claim 1, wherein the at least one triglyceride of at least one hydroxylated carboxylic acid is chosen from triglycerides of at least one hydroxylated carboxylic acid wherein the at least one hydroxylated carboxylic acid comprises from 6 to 40 carbon atoms.

4. The composition according to claim 3, wherein the at least one hydroxylated carboxylic acid comprises from 10 to 34 carbon atoms.

5. The composition according to claim 4, wherein the at least one hydroxylated carboxylic acid comprises from 12 to 28 carbon atoms.

6. The composition according to claim 5, wherein the at least one hydroxylated carboxylic acid comprises from 16 to 20 carbon atoms.

7. The composition according to claim 6, wherein the at least one hydroxylated carboxylic acid comprises 18 carbon atoms.

8. The composition according to claim 1, wherein the at least one hydroxylated carboxylic acid is chosen from:
i) saturated linear monohydroxylated aliphatic monocarboxylic acids of formulae (1) and (2)
(1) 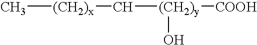
with 3≦x+y≦37
and
HO—CH₂—(CH₂)ₓ—COOH (2)
with 4≦x≦38;
ii) saturated branched monohydroxylated aliphatic monocarboxylic acids of formulae (3) and (3′)
(3) 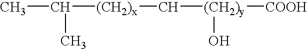
with 1≦x+y≦35
and (3′) 2-ethyl-3-hydroxycaprylic acid of formula 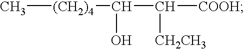
iii) unsaturated monohydroxylated aliphatic monocarboxylic acids of formulae (4), (5), and (6)
(4) 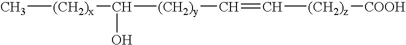
with 1≦x+y+z≦35
(5) 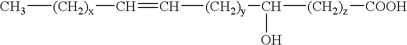
with 1≦+y+z≦35
and
HOCH₂—(CH₂)ₓ—CH═CH—(CH₂)_{y}—COOH (6)
with 2≦x+y≦36;
iv) saturated polyhydroxylated aliphatic monocarboxylic acids of formula (7)
(7) 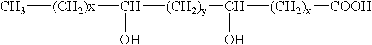
with 2≦x+y+z≦36
and the corresponding unsaturated polyhydroxylated aliphatic monocarboxylic acids;
v) saturated monohydroxylated aliphatic polyacids of formula (8)
(8) 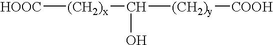
with 3≦x+y≦37
and the corresponding unsaturated monohydroxylated aliphatic polyacids; and
vi) saturated and unsaturated polyhydroxylated aliphatic polyacids.

9. The composition according to claim 8, wherein the at least one hydroxylated carboxylic acid is chosen from
12-hydroxystearic acid, α-hydroxyoctadecanoic acid, hydroxy-14-eicosenoic acid;
leucinic acid, 2-ethyl-3-hydroxycaprylic acid;
ricinoleic acid;
3-hydroxy-4-hexanoic acid, oxynervonic acid;
16-hydroxy-6-hexadecenoic acid;
9,10-dihydroxyoctadecanoic acid, 9,12-dihydroxyoctadecanoic acid, aleuritic acid, 9,10,12-trihydroxyoctadecanoic acid, hexahydroxyoctadecanoic acid and octahydroxyoctadecanoic acid.

10. The composition according to claim 9, wherein the at least one triglyceride of at least one hydroxylated carboxylic acid is a triglyceride of ricinoleic acid.

11. The composition according claim 1, wherein the esterification of said at least one triglyceride of at least one hydroxylated carboxylic acid is performed with at least one aliphatic monocarboxylic acid comprising from 6 to 40 carbon atoms.

12. The composition according to claim 11, wherein the at least one aliphatic monocarboxylic acid comprises from 10 to 34 carbon atoms.

13. The composition according to claim 12, wherein the at least one aliphatic monocarboxylic acid comprises from 12 to 28 carbon atoms.

14. The composition according to claim 13, wherein the at least one aliphatic monocarboxylic acid comprises from 16 to 20 carbon atoms.

15. The composition according to claim 14, wherein the at least one aliphatic monocarboxylic acid comprises 18 carbon atoms.

16. The composition according to claim 11, wherein the at least one aliphatic monocarboxylic acid is chosen from saturated and unsaturated aliphatic fatty acids.

17. The composition according to claim 16, wherein the aliphatic fatty acid is isostearic acid.

18. The composition according to claim 1, wherein the at least one aliphatic dicarboxylic acid comprises from 3 to 10 carbon atoms.

19. The composition according to claim 18, wherein the at least one aliphatic dicarboxylic acid comprises from 3 to 6 carbon atoms.

20. The composition according to claim 18, wherein the at least one aliphatic dicarboxylic acid is chosen from aliphatic dicarboxylic acids of formula HOOC—(CH₂)ₙ—COOH wherein n=1 to 4.

21. The composition according to claim 20, wherein the at least one a!iphatic dicarboxylic acid is succinic acid of formula HOOC—(CH₂)ₙ—COOH wherein n=2.

22. The composition according to claim 1, wherein the at least one polyester is of the formula (I)
T₂O—(OC-D-CO—O-T₁-O)ₓ—OC-D-CO-OT₂ (I)
wherein
T₂-O— originates from compound T₂-OH, which is a triglyceride of at least one hydroxylated carboxylic acid comprising a single free hydroxyl functional group;
—O-T₁-O— originates from compound HO-T₁-OH, which is a triglyceride of at least one hydroxylated carboxylic acid comprising two free hydroxyl functional groups;
—OC-D-CO— originates from compound HOOC-D-COOH, which is said at least one aliphatic dicarboxylic acid, and
x ranges from 1 to 50.

23. The composition according to claim 22, wherein x ranges from 1 to 10.

24. The composition according to claim 23, wherein x ranges from 2 to 6.

25. The composition according to claim 22, wherein the compound T₂-OH is a triglyceride of at least one hydroxylated carboxylic acid, and said triglyceride is esterified with two molecules of the at least one aliphatic monocarboxylic acid.

26. The composition according to claim 22, wherein the compound HO-T₁-OH is a triglyceride of at least one hydroxylated carboxylic acid, and said triglyceride is esterified with one molecule of the at least one aliphatic monocarboxylic acid.

27. The composition according to claim 22, wherein the at least one polyester is of the formula (I) wherein
T₂O is 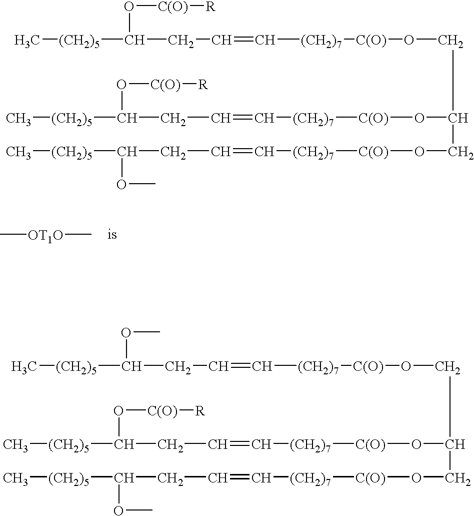
wherein R is chosen from alkyl and alkylene groups comprising from 5 to 33 carbon atoms.

28. The composition according to claim 27, wherein R is chosen from alkyl groups comprising from 7 to 17 carbon atoms.

29. The composition according to claim 27, wherein R is chosen from alkylene groups comprising from 11 to 21 carbon atoms.

30. The composition according to claim 1, wherein the at least one polyester is liquid at ambient temperature and atmospheric pressure.

31. The composition according to claim 30, wherein the at least one polyester has a viscosity of more than 500 cP (50 Pa.s) at 25° C. and/or a refractive index ≧1.47.

32. The composition according to claim 31, wherein the at least one polyester has a viscosity ranging from 900 to 10 000 cP (90 to 1 000 Pa.s) at 25° C.

33. The composition according to claim 32, wherein the at least one polyester has a viscosity ranging from 950 to 5 000 cP (95 to 500 Pa.s) at 25° C.

34. The composition according to claim 31, wherein the at least one polyester has a refractive index ranging from 1.47 to 1.55.

35. The composition according to claim 34, wherein the at least one polyester has a refractive index ranging from 1.48 to 1.55.

36. The composition according to claim 1, wherein the at least one polyester is present in an amount sufficient to endow the composition with at least one property chosen from properties of slip, gloss, colour stability and colour retention over time, gloss retention over time, comfort, non-migration and outline definition of the applied said composition.

37. The composition according to claim 1, wherein the at least one polyester is present in an amount ranging from 0.1% to 99.9% by weight of the total weight of the composition.

38. The composition according to claim 37, wherein the at least one polyester is present in an amount ranging from 10% to 40% by weight of the total weight of the composition.

39. The composition according to claim 38, wherein the at least one polyester is present in an amount ranging from 15% to 25% by weight of the total weight of the composition.

40. The composition according to claim 39, wherein the at least one polyester is present in an amount ranging from 20% to 25% by weight of the total weight of the composition.

41. The composition according to claim 1, wherein the at least one oil has a molar mass ranging from 750 to 7 500 g/mol.

42. The composition according to claim 1, wherein the at least one oil is chosen from
lipophilic polymers;
linear fatty acid esters comprising a total carbon number ranging from 35 to 70;
hydroxylated esters;
aromatic esters;
C₂₄-C₂₈ branched fatty acid and fatty alcohol esters;
silicone oils; and
oils of vegetable origin.

43. The composition according to claim 1, wherein the at least one oil is chosen from polybutylenes, hydrogenated polyisobutylenes, polydecenes, hydrogenated polydecenes, vinylpyrrolidone copolymers, pentaerythrityl tetrapelargonate, polyglycerol-2 triisostearate, tridecyl trimellitate, triisoarachidyl citrate, pentaerythrityl tetraisononanoate, glyceryl triisostearate, glyceryl 2-tridecyltetradecanoate, pentaerythrityl tetraisostearate, polyglyceryl-2 tetraisostearate, pentaerythrityl 2-tetradecyltetradecanoate, phenylsilicones, and sesame oil.

44. The composition according to claim 43, wherein the vinylpyrrolidone copolymers are chosen from PVP/hexadecene copolymers.

45. The composition according to claim 1, wherein the at least one oil is present in an amount ranging from 1% to 99% by weight of the total weight of the composition.

46. The composition according to claim 45, wherein the at least one oil is present in an amount ranging from 10% to 80% by weight of the total weight of the composition.

47. The composition according to claim 45, wherein the at least one oil is present in an amount ranging from 5% to 70% by weight of the total weight of the composition.

48. The composition according to claim 1, wherein the composition is in a form chosen from lipsticks, lipglosses, blushers, eyeshadows, mascaras, eyeliners, nail varnishes, artificial skin tanning products, haircare products, hair coloring products.

49. The composition according to claim 1, wherein the at least one colorant is chosen from dyes, which are soluble or dispersible in the composition, pigments, and nacres.

50. The composition according to claim 1, further comprising at least one wax chosen from polyethylene waxes with a molecular weight ranging from 400 to 800 g/mol.

51. The composition according to claim 1, wherein the composition is in a form chosen from cast and compacted forms.

52. The composition according to claim 1, wherein the composition is in an anhydrous form.

53. The composition according to claim 48, wherein the composition is in a form of a lipstick or lipgloss.

54. A method of endowing a film of a cosmetic composition with at least one:
property chosen from good application properties, good properties of spreading, of colour retention following challenge, of comfort (no tightening or drying out) and of non-migration, with outlines which, when deposited on a keratin material, are well defined, and with enhanced color intensity, comprising including in said cosmetic composition
i) at least one polyester resulting from esterification of at least one triglyceride of at least one hydroxylated carboxylic acid with at least one aliphatic monocarboxylic acid and at least one aliphatic dicarboxylic acid,
ii) at least one oil of a molar mass ranging from 650 to 10 000 g/mol, and
iii) at least one colorant.

55. A method of endowing a composition with at least one property chosen from good application properties and good properties of spreading, of definition, of comfort (no tightening or drying out) and of non-migration; with enhanced color intensity; and with enhanced color retention after challenge, comprising including in the composition
i) at least one polyester resulting from esterification of at least one triglyceride of at least one hydroxylated carboxylic acid with at least one aliphatic monocarboxylic acid and at least one aliphatic dicarboxylic acid and,
ii) at least one oil of a molar mass ranging from 650 to 10 000 g/mol,
wherein the composition comprises, in a physiologically acceptable medium, at least one colorant.

56. A method of making a cosmetic care and/or makeup composition for a keratin material, comprising, including in the composition
i) at least one polyester resulting from esterification of at least one triglyceride of at least one hydroxylated carboxylic acid with at least one aliphatic monocarboxylic acid and at least one aliphatic dicarboxylic acid,
ii) at least one oil of a molar mass ranging from 650 to 10 000 g/mol, and
iii) at least one colorant.
